(19) United States
(12) Patent Application Publication
(10) Pub. No.: US 2026/0134440 A1
(43) Pub. Date: May 14, 2026

(54) AUTOMATED QUALITY ASSURANCE AUDITING ASSISTANT (AQAAA)

(71) Applicant: Emil Anthony Kamar, Raleigh, NC (US)

(72) Inventor: Emil Anthony Kamar, Raleigh, NC (US)

(21) Appl. No.: 19/358,500

(22) Filed: Oct. 15, 2025

Related U.S. Application Data
(60) Provisional application No. 63/720,705, filed on Nov. 14, 2024.

(51) Int. Cl. G06Q 30/018; G16H 10/20; G16H 10/60; G16H 40/20

(52) U.S. Cl. CPC G06Q 30/018; G16H 10/20; G16H 10/60; G16H 40/20

(57) ABSTRACT

The disclosed system provides an adaptive, modular platform for automated compliance in clinical research and other environments. It integrates a rule-based artificial intelligence engine, real-time sensor feedback, and blockchain-based audit logging to ensure protocol adherence and regulatory transparency. The system supports deployment across cloud, edge, and hybrid infrastructures, enabling contextual enforcement through non-persistent data processing and immutable event tracking. By dynamically interpreting trial protocols and adjusting enforcement logic based on environmental inputs, the system facilitates continuous compliance with HIPAA, 21 CFR Part 11, Good Clinical Practice (GCP), protocol, and SOP standards. The architecture is designed for scalability, auditability, and proactive corrective action planning across diverse trial sites and study phases.

### RELATED PROVISIONAL APPLICATION

Application No. 63/720,705

Filing Date: 14 Nov. 2024

Applicant: Emil Anthony Kamar

### FIELD

The present application relates to a system and method for automated compliance auditing using artificial intelligence, modular architecture, and immutable audit trails.

### BACKGROUND

Clinical research, laboratory operations, and manufacturing systems are subject to strict regulatory oversight, governed by frameworks such as 21 CFR Parts 11, 50, 54, and 56, the International Council for Harmonisation (ICH) Good Clinical Practice (GCP) guidelines, the Declaration of Helsinki, ISO standards, and related local and international laws. Quality assurance and auditing in these environments have historically relied on manual processes performed by human auditors. These processes involve the review of study protocols, source data, informed consent forms, adverse event reports, and regulatory documentation. Manual auditing is inherently limited by the availability of human expertise, variability in interpretation, and significant time delays between data generation and audit resolution.

Existing electronic solutions—including electronic medical record (EMR) systems, electronic data capture (EDC) platforms, and eRegulatory tools—have improved documentation but remain siloed and fragmented. Interoperability between systems is inconsistent, requiring auditors to cross-reference multiple data sources manually. Furthermore, most available audit tools function retrospectively, identifying issues only at interim analyses or after study completion, when corrective actions are costly and regulatory risk is heightened.

Several technical limitations persist in the prior art:
Latency—Existing auditing solutions lack real-time validation, introducing delays in identifying protocol deviations, safety concerns, or consent irregularities.
Interoperability—Current platforms struggle to seamlessly integrate heterogeneous data sources (e.g., EMRs, EDCs, laboratory information systems), forcing reliance on manual reconciliation.
Predictive Capability—Conventional auditing systems are reactive and do not employ machine learning or predictive analytics to anticipate compliance risks.
Audit Integrity—Digital audit trails are often modifiable or incomplete, undermining confidence in data integrity and traceability.
Scalability—Most systems cannot adapt dynamically to changes in study protocols, site standard operating procedures (SOPs), or evolving regulatory frameworks.

These limitations increase regulatory exposure, delay the detection of non-compliance, and elevate operational costs. There is therefore a pressing need for a technically advanced compliance auditing system that:
Performs near real-time data validation across multiple data sources;
Provides secure, immutable auditability resistant to tampering;
Uses artificial intelligence to interpret study protocols, adapt enforcement logic dynamically, and detect patterns of potential non-compliance;
Incorporates predictive analytics informed by historical regulatory enforcement actions to mitigate risk proactively; and
Scales across multi-site clinical programs while maintaining data privacy and regulatory compliance.

The present invention, the Automated Quality Assurance Auditing Assistant (AQAAA), addresses these deficiencies by introducing a modular, AI-driven platform with secure interoperability protocols and immutable blockchain-based audit trails. AQAAA advances the state of compliance technology by transforming auditing from a retrospective, manual process into a real-time, adaptive, and verifiable system that enhances efficiency, accuracy, and ethical oversight in regulated research and manufacturing environments.

### SUMMARY

The present invention provides a computer-implemented system and method for automated quality assurance and compliance auditing in regulated environments, including but not limited to clinical research, laboratory operations, and pharmaceutical manufacturing. The system integrates artificial intelligence, modular architecture, and immutable audit logging to deliver near real-time compliance validation, risk prediction, and corrective action support across distributed and heterogeneous data sources.

In one aspect, the invention comprises a protocol interpretation engine that transforms textual trial protocols into executable, machine-readable rules using natural language processing and rule-based artificial intelligence. This enables automated enforcement of study-specific requirements and regulatory standards, including 21 CFR Part 11, ICH GCP, and related guidelines.

In another aspect, the invention includes a machine learning compliance engine configured to ingest data from multiple sources—such as electronic medical records (EMRs), electronic data capture (EDC) platforms, laboratory information systems, and connected sensors—through secure application programming interfaces (APIs). The engine performs real-time data comparisons against protocol-derived rules, identifies discrepancies or deviations, and generates risk-weighted alerts for auditor review.

In yet another aspect, the invention provides a predictive risk scoring module trained on historical regulatory enforcement actions, including U.S. Food and Drug Administration (FDA) Form 483 observations and warning letters. This module categorizes discrepancies by severity and likelihood of regulatory consequence, enabling proactive risk mitigation and prioritization of corrective actions.

All compliance events, alerts, and user interactions are recorded in an immutable blockchain-based audit ledger, which employs cryptographic integrity mechanisms, such as Merkle tree structures and zero-knowledge proofs, to ensure tamper resistance while preserving patient confidentiality.

The modular system design further includes a deployment framework that supports cloud-based, edge-based, or hybrid operation, with containerized modules for Good Clinical Practice (GCP), Good Laboratory Practice (GLP), Good Manufacturing Practice (GMP), Institutional Review Board (IRB) oversight, vendor compliance, and financial auditing. This allows for flexible customization to meet site-specific standard operating procedures (SOPs), sponsor requirements, and evolving regulatory changes.

The invention also provides a user interface and visualization suite that delivers:
Customizable dashboards displaying near real-time compliance status;
Automated generation of audit reports with regulatory citations;
Corrective action recommendations and tracking tools; and
Trend analyses of recurring compliance risks across studies and sites.

Through this combination of AI-driven automation, predictive analytics, modular deployment, and immutable recordkeeping, the invention advances the state of compliance auditing. It reduces latency in identifying protocol deviations, enhances audit integrity, scales efficiently across multi-site programs, and enables both retrospective and near real-time oversight. As such, the invention delivers measurable improvements in efficiency, accuracy, and regulatory compliance assurance beyond what is achievable with existing manual or semi-automated auditing systems.

### DETAILED DESCRIPTION

The Automated Quality Assurance Auditing Assistant (AQAAA) is implemented as a modular, computer-implemented platform comprising:
1. Protocol Interpretation Engine (PIE)—configured to parse study protocols written in natural language and convert them into machine-executable compliance rules. The engine applies natural language processing (NLP) and a rule-based classifier to identify critical requirements such as inclusion/exclusion criteria, informed consent conditions, safety reporting obligations, and investigational product handling instructions.
2. Machine Learning Compliance Engine (MLCE)—configured to ingest structured and unstructured data from multiple sources, including electronic medical records (EMRs), electronic data capture (EDC) platforms, laboratory information systems (LIMS), and sensor-enabled devices (e.g., temperature monitors, wearable patient trackers). Data ingestion occurs through secure APIs without persistent storage to preserve patient confidentiality. The MLCE compares incoming data to protocol-derived rules, flags discrepancies, and generates risk-weighted alerts.
3. Predictive Risk Scoring Module (PRSM)—trained on datasets of historical regulatory enforcement actions, including U.S. Food and Drug Administration (FDA) Form 483s and warning letters. The PRSM applies statistical modeling and machine learning (e.g., logistic regression, gradient boosting) to assign severity scores to detected deviations based on their frequency and regulatory impact in prior cases.
4. Immutable Audit Ledger—implemented using a blockchain-based architecture with Merkle tree structures to ensure cryptographic integrity. All alerts, risk scores, user interactions, and corrective action records are immutably stored. The ledger optionally employs zero-knowledge proofs to validate compliance without exposing sensitive patient identifiers.
5. Deployment Framework—comprising a containerized, microservices architecture that supports operation in cloud-based, edge-based, or hybrid environments. Each compliance module (e.g., GCP, GLP, GMP, IRB, vendor, budget) may be deployed independently or in combination.
6. User Interface and Visualization Suite—providing customizable dashboards, automated audit reports with regulatory citations, corrective action tracking tools, and trend analysis visualizations.

### Functional Modules

### Protocol Interpretation Engine (PIE)

The PIE receives protocol text in formats such as PDF or Word documents. Using NLP pipelines, it identifies key regulatory, or rules statements, such as:
“Informed consent must be obtained before the administration of the first investigational dose.”
“Temperature excursions beyond 2-8° C. must be reported within 24 hours.”

The system converts these into executable rules. For example:
Rule 1: consent_timestamp<first_dose_timestamp
Rule 2: (temp_reading<2 OR temp_reading>8)→report_within(24 h)

These rules are stored in a dynamic rule library, enabling the compliance engine to validate real-world data automatically.

### Machine Learning Compliance Engine (MLCE)

The MLCE continuously receives structured records (e.g., EMR timestamps, EDC entries) and unstructured data (e.g., scanned consent forms processed via OCR). It applies rule-matching logic and anomaly detection models.
If data aligns with protocol rules→marked compliant.
If deviations are detected→generates risk-weighted alerts routed to the dashboard.

The MLCE includes a feedback loop: auditor inputs (e.g., “false positive,” “valid deviation”) are stored and used to refine model thresholds and rule-matching confidence.

### Predictive Risk Scoring Module (PRSM)

The PRSM evaluates flagged discrepancies against a knowledge base of historical FDA findings. For example, deviations involving incomplete informed consent are weighted as high-risk due to frequent regulatory citations, whereas minor laboratory sample mislabels may be moderate risk.

Severity scores are displayed on the dashboard using a three-tier classification:
High risk (May involve direct risk to participant safety or data integrity, or likely to trigger regulatory action).
Moderate risk (requires corrective action but less likely to result in citations).
Low risk (document and monitor).

### Immutable Audit Ledger

All events are written to a blockchain ledger:
Input events (e.g., data ingestion timestamps).
Alerts (e.g., consent discrepancy flagged).
User interactions (e.g., auditor overrides).
Corrective actions (e.g., retraining staff, updating SOPs).

Merkle tree hashing ensures tamper resistance, and zero-knowledge proof protocols allow regulators to validate compliance events without accessing raw patient data.

### User Interface

The user interface includes:
Dashboard with risk-prioritized alerts.
Audit Report Generator producing regulatory citations linked to specific findings.
Corrective Action Tracker logging remediation status.
Trend Analysis Visualization identifying recurring issues across studies/sites.

### Worked Example 1: Informed Consent Validation

1. The PIE parses protocol text: “Informed consent must be signed prior to first dose.”
2. Executable rule generated: consent_timestamp<first_dose_timestamp.
3. MLCE ingests EMR record showing consent_timestamp=09:00 and first_dose_timestamp=08:45.
4. Rule violation detected.
5. Alert generated with risk score=High, based on FDA enforcement history of consent violations.
6. Event logged immutably to blockchain.
7. Dashboard displays corrective action recommendation: “Conduct staff retraining on consent procedures.”

### Worked Example 2: Investigational Product Accountability

1. Protocol requires: “Investigational product must be stored at 2-8° C. Excursions must be reported within 24 hours.”
2. PIE generates rule: (temp_reading<2 OR temp_reading>8)→report_within(24 h).
3. MLCE ingests IoT temperature sensor data: temp_reading=12° C. at 14:00.
4. No record of report filed within 24h detected in EDC system.
5. Deviation flagged.
6. PRSM assigns risk score=Moderate (frequent but less severe than consent deviations).
7. Blockchain logs excursion event, non-reporting, and auditor override.
8. Dashboard trend analysis shows repeated temperature excursions across three sites→flagged as systemic risk.

### Advantages of the Invention

Reduced latency—deviations detected within minutes rather than weeks.
Improved audit integrity—blockchain ensures tamper-proof audit trails.
Scalability—containerized modules adapt to evolving regulatory requirements, then scalable across multiple sites.
Predictive oversight—risk scoring anticipates likely regulatory findings.
Operational efficiency—automated corrective action tracking reduces auditor workload.

### BRIEF DESCRIPTION OF THE DRAWINGS

In the following detailed portion of the present description, the teachings of the present application will be explained in more detail with reference to the example embodiments shown in the illustrations, in which:

FIG. 1 is a modular system architecture overview for the Automated Quality Assurance Auditing Assistant (AQAAA) with sensory feedback loops.

FIG. 2 is a workflow diagram showing data flow and audit operations in the AQAAA system.

FIG. 3 is an example user interface dashboard presenting risk-weighted alerts and corrective action tracking.

FIG. 4 is a trend analysis visualization illustrating deviation patterns across multiple sites over time.

FIG. 5 is a security flow model depicting encrypted input, processing, immutable logging, and compliance verification zones.

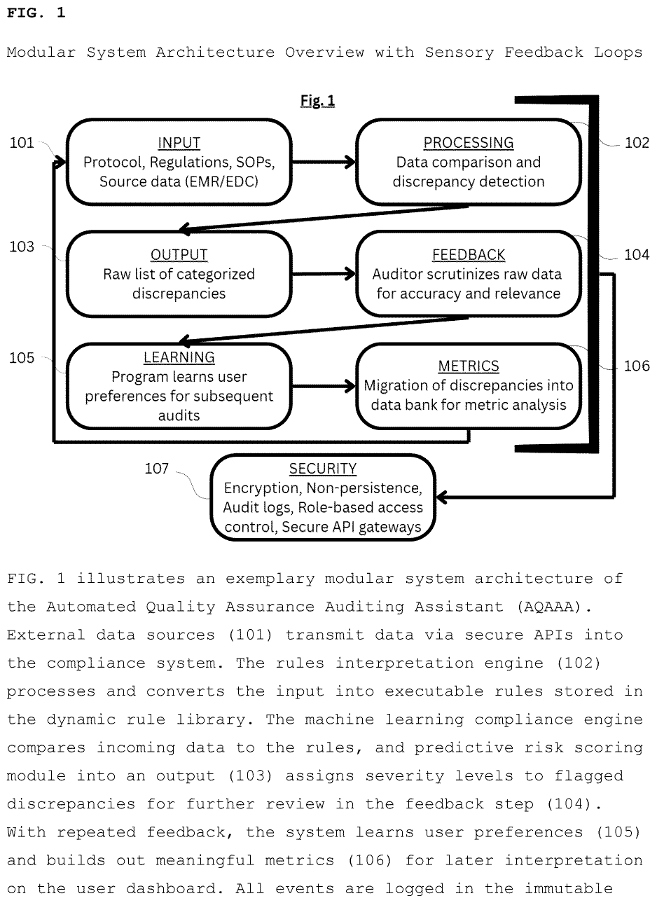

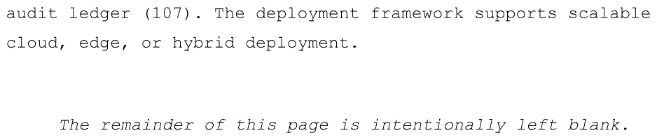

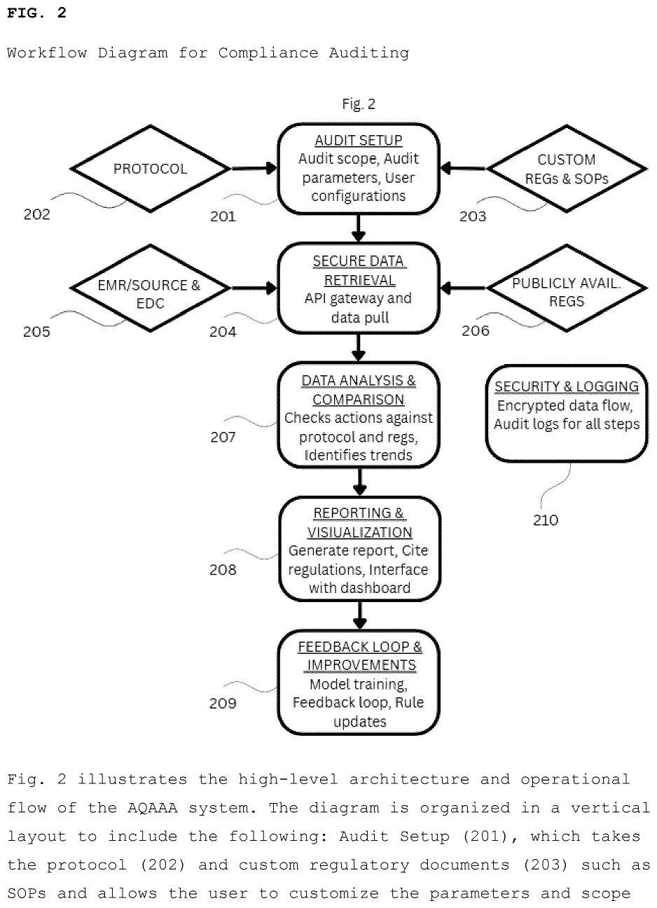

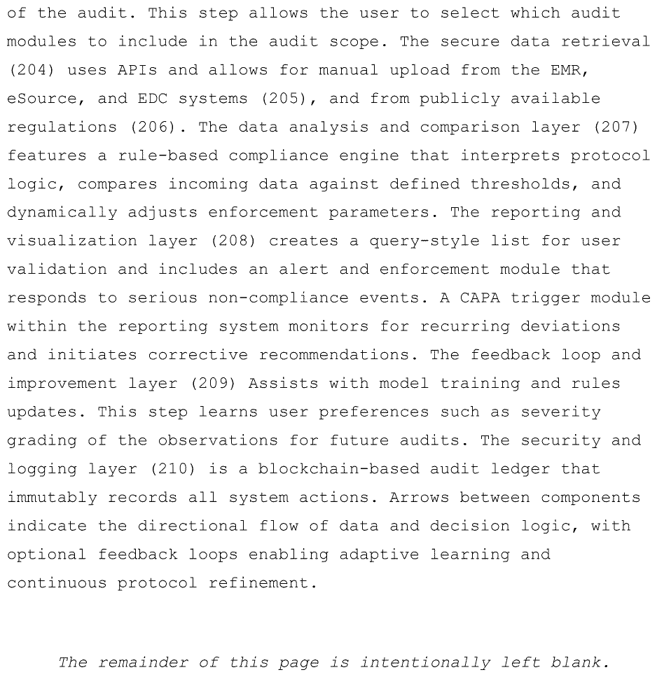

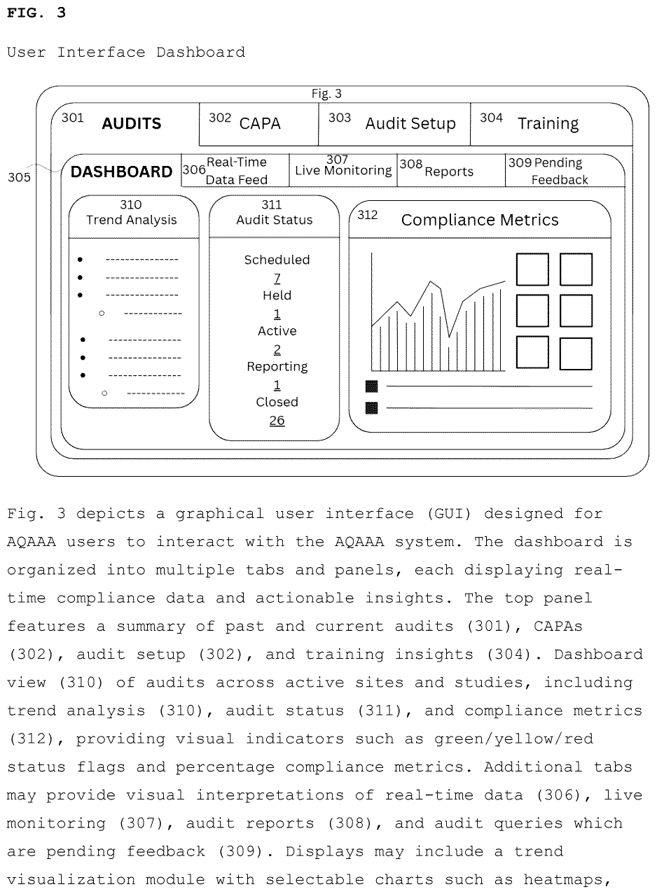

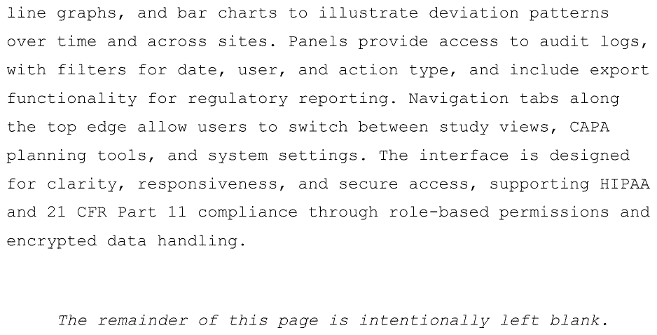

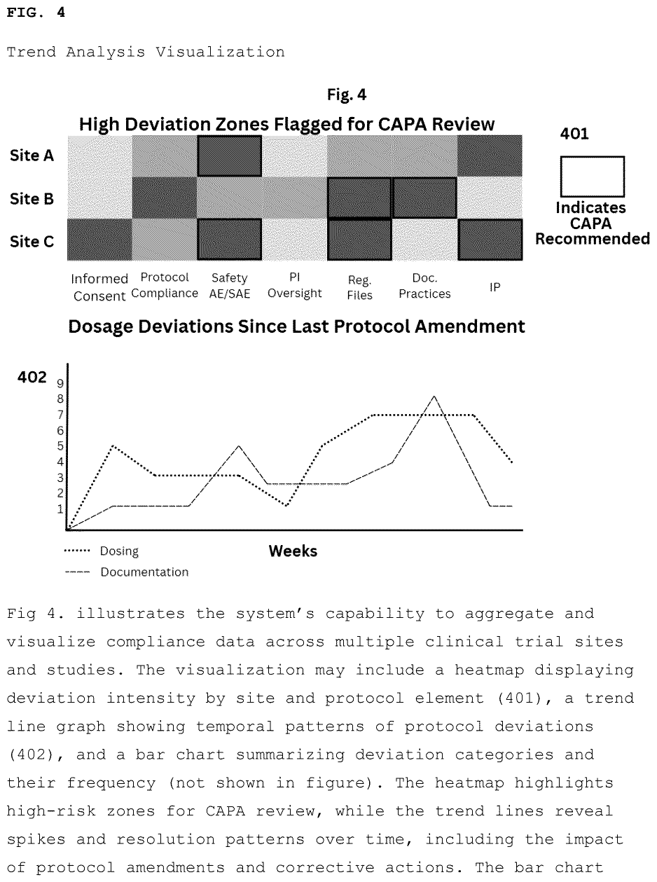

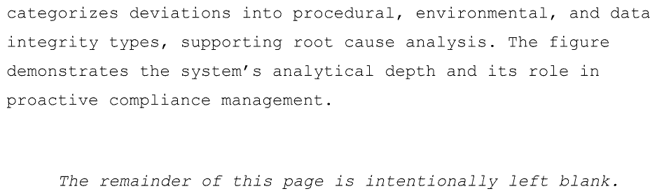

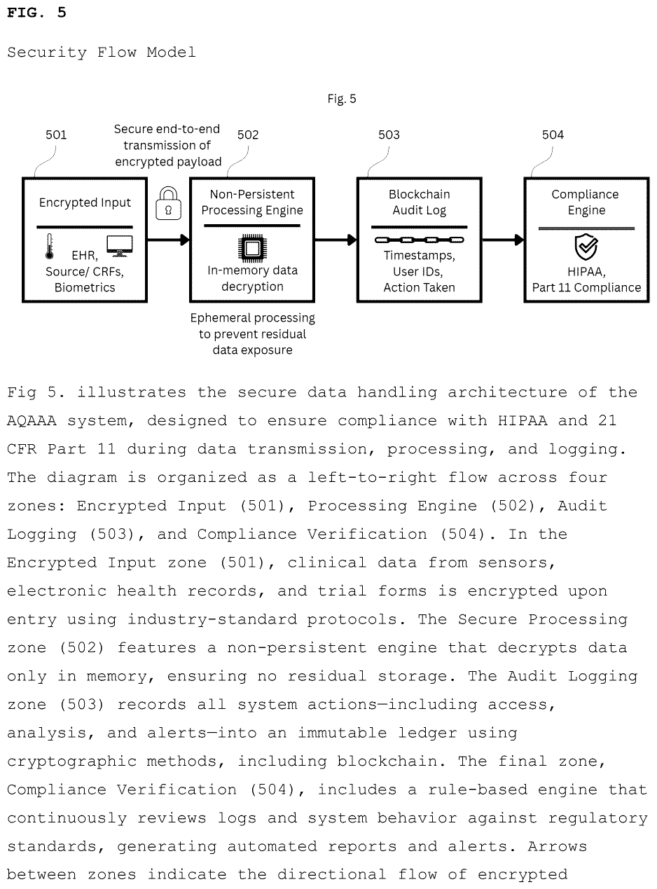

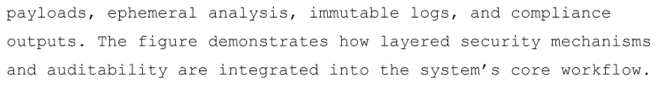

### DEFINITIONS SECTION (CLARIFYING FUNCTIONAL LANGUAGE)

For clarity and consistency, the following terms are defined as they are used within this specification. These definitions are intended to capture the functional and structural scope of the invention and to ensure that the terms are not limited by narrower meanings in prior art.

thresholds and improve accuracy over time.

trail records.

“Risk-Based Monitoring Plan”

What is claimed is:

1) A computer-implemented system for automated compliance auditing in clinical research, laboratory, and manufacturing environments, the system comprising a modular architecture configured with distinct compliance modules for Good Clinical Practice (GCP), Good Laboratory Practice (GLP), Good Manufacturing Practice (GMP), Institutional Review Board oversight, vendor compliance, and financial and budget compliance; a protocol interpretation engine configured to transform textual trial protocols into executable machine-readable rules using natural language processing and rule-based artificial intelligence; a machine-learning compliance engine configured to receive real-time data from heterogeneous sources including electronic medical records, electronic data capture platforms, and environmental sensors, compare the received data to the executable rules, detect discrepancies, deviations, or potential noncompliance events, and generate risk-weighted alerts in real time; a predictive risk scoring module trained on historical regulatory enforcement actions to assign severity levels to detected discrepancies; a blockchain-based audit ledger configured to immutably record alerts, risk scores, and corrective actions using a Merkle tree data structure; a deployment framework operable in cloud, edge, or hybrid environments with fallback to manual logging; and a user interface configured to present customizable dashboards, corrective action recommendations, and trend analysis visualizations.

2) The system of claim 1, wherein the compliance engine includes a feedback loop that dynamically adjusts enforcement thresholds based on historical data patterns and auditor input.

3) The system of claim 1, wherein the compliance engine comprises a plurality of artificial intelligence agents each specialized in a compliance domain selected from regulatory documentation, informed consent, investigational product accountability, safety event monitoring, staff delegation tracking, and other compliance areas.

4) The system of claim 1, wherein the predictive risk scoring module automatically generates site-specific risk-based monitoring plans.

5) The system of claim 1, wherein the predictive risk scoring module incorporates statistical trends derived from US Food and Drug Administration Form 483s and warning letters from the five years prior to system execution.

6) The system of claim 1, wherein the blockchain-based audit ledger applies a zero-knowledge proof protocol to verify data integrity without exposing patient identifiers.

7) The system of claim 1, wherein the user interface further tracks corrective action implementation progress and resolution status.

8) The system of claim 1, wherein the compliance modules are containerized and user-selectable to allow customization for study-specific or sponsor-specific requirements.

9) The system of claim 1, wherein the user interface generates automated trend analyses of recurring compliance issues across multiple sites and time periods.

10) The system of claim 1, wherein data ingestion occurs exclusively through secure application programming interfaces without persistent storage, thereby maintaining HIPAA compliance and patient privacy.
